(19) United States
(12) Patent Application Publication
(10) Pub. No.: US 2026/0345537 A1
(43) Pub. Date: Nov. 5, 2026

(54) SUSTAINED RELEASE PHARMACEUTICAL COMPOSITIONS COMPRISING PREGABALIN OR PHARMACEUTICALLY ACCEPTABLE SALTS THEREOF

(71) Applicant: ORBIT PHARMA LIMITED, London (GB)

(72) Inventor: Hetalbahen Patel, London (GB)

(21) Appl. No.: 19/116,277

(22) PCT Filed: Nov. 28, 2023
(86) PCT No.: PCT/GB2023/053074

§ 371 Date: Mar. 27, 2025

(30) Foreign Application Priority Data
Nov. 28, 2022 (GB) 2217820.6

(51) Int. Cl. A61K 31/197; A61K 9/20; A61K 9/28

(52) U.S. Cl. CPC A61K 31/197; A61K 9/2009; A61K 9/2013; A61K 9/2018; A61K 9/2027; A61K 9/2054; A61K 9/2095; A61K 9/2813; A61K 9/284; A61K 9/2853; A61K 9/2893

(57) ABSTRACT

The present invention relates to a stable once-daily sustained release film-coated tablet comprising pregabalin or pharmaceutically acceptable salts thereof, a combination of at least two release retarding agents, a diluent, and a lubricant. The present invention provides a uniform release rate of the drug, improves patient compliance by reducing the frequency of drug administration.

### FIELD OF THE INVENTION

The present invention relates to a pharmaceutical composition of Pregabalin. The present invention relates to a sustained release film-coated tablet of Pregabalin or pharmaceutically acceptable salts thereof, suitable for once-daily oral administration. The present invention also relates to the process of the preparation of the same.

### BACKGROUND OF THE INVENTION

Pregabalin was first disclosed in the U.S. Pat. No. 6,197,819. Pregabalin is also called β-isobutyl-γ-aminobutyric acid or isobutyl-GABA. Pregabalin is a structural derivative of the inhibitory neurotransmitter GABA, however, it does not bind to GABAA, GABAB, or benzodiazepine receptors and does not enhance GABAA responses in cultured neurons. Pregabalin, however, enhances the density of GABA transporter protein and increases the rate of functional GABA transport in cultured neurons when it is administered for a prolonged period of time. When the concentration of GABA diminishes below a threshold level appears to terminate seizures.

Pregabalin inhibits the calcium-dependent release of pro-nociceptive neurotransmitters in the spinal cord, possibly by interfering with the movement of calcium channels that contain alpha2-delta and reducing calcium currents. The anti-nociceptive effects of pregabalin may interact with descending noradrenergic and serotonergic pathways, which originate in the brainstem and influence spinal cord pain transmission. pregabalin is indicated for the treatment of postherpetic neuralgia, fibromyalgia, and as adjunctive therapy for the treatment of partial-onset seizures in adults. It is also indicated for the management of neuropathic pain related to spinal cord injury and diabetic peripheral neuropathy or generalized anxiety disorder.

The IUPAC name of pregabalin is (3S)-3-(aminomethyl)-5-methylhexanoic acid. Pregabalin is marketed as an oral tablet, capsule, solution, and powder. The commercially marketed products of pregabalin in tablet form are available in three dosage strengths: 82.5 mg, 165 mg, and 330 mg for oral administration. The dosage strength of the Pregabalin capsule is available in 25, 50, 75, 100, 150, 200, 225, and 300 mg capsules, as well as in an oral solution containing 20 mg/mL. Pregabalin oral bioavailability is reported to be >90% regardless of the dose. The elimination half-life of pregabalin is 6.3 hours. The mean renal clearance is estimated to be 67.0 to 80.9 mL/min.

U.S. Pat. No. 8,945,620 B2 discloses an oral tablet containing pregabalin, matrix forming agent including mixtures of polyvinyl acetate and polyvinylpyrrolidone, and the swelling agent includes cross-linked polyvinylpyrrolidone and polyethylene oxide. The Pregabalin was dry blended with matrix-forming agents, followed by the addition of other excipients. To prepare the final drug product, the compressed dosage forms may undergo further processing such as polishing and coating.

WO 2010/115612A2 discloses an oral capsule that comprises 100 parts of pregabalin and 15 parts of pregelatinized starch, wherein said blend is free of lactose, mannitol, or microcrystalline cellulose. The process comprises sieving and blending the pregabalin with pregelatinized starch to a homogenous blend. To the blend, talc was added and the mixture was blended again. The final mixture was filled in a size 0 capsule in an amount of 400 mg of the blend per capsule.

WO 2013/100874 A1 discloses an effervescent powder, tablet, and granule comprising of pregabalin and at least one pharmaceutically acceptable excipient selected from the effervescent couple, flavoring agent, binder, lubricant, sweetener, and taste regulating agent. The effervescent formulation was prepared by the wet granulation method. The process of this invention involves mixing a binder, solvent, and active agent to prepare a granulation solution. Mix pregabalin with another pharmaceutically acceptable excipient. Wet granulating the mixture with the granulation solution and then granules are dried and sieved. The granules are compressed into a tablet, bottle-filled, or sachet filled to produce the specified dosage form.

US 2005/0171203A1 discloses the oral liquid pharmaceutical composition of pregabalin contains at least each preservative, taste-masking agent, and viscosity-controlling agent. The process involves adding sterile water to a vessel and then the contents of the vessel are heated to 80 °C. Methylparaben and ethylparaben is stirred into the hot water. After a clear solution is obtained, Sodium Saccharin and hydroxyethylcellulose are added. The resulting liquid mixture is cooled to 30 °C. The pregabalin and flavoring agent are added in small portions with Stirring. The solution is filtered using a Millipore® membrane filter and Stored in Sealed containers.

WO 2009/087682A2 discloses an injectable formulation of pregabalin which is stable between pH 4.0 to 8.0. The process involves taking water for injection into an SS container and sparge with 0.2μ filtered nitrogen until the level of dissolved oxygen falls below 1 ppm. Dissolve sodium chloride, citric acid monohydrate, and pregabalin with continuous nitrogen sparging. Make up the volume with nitrogen-sparged water for injection. Filter the bulk solution through a 0.2μ polyvinylfluoridine filter by using nitrogen gas. Fill the filtered bulk solution into 5 ml clear glass USP type 1 ampoules.

The present invention of pregabalin or pharmaceutically acceptable salts thereof provides sustained release film-coated tablet composition for once-daily (QD) dosing. When administered in a solid dosage form, the Pregabalin continuously releases while being retained in the stomach for a longer period of time. In contrast, the liquid dosage form is bulky, difficult to transport, and takes up a lot of space. Due to their inherent instability, liquid dosage forms often have shorter shelf lives. The patient's measurement of the exact volume determines whether the dose is administered appropriately, which increases the chance for variability.

The dosing regimen of 2-3 daily doses is problematic which results from the sharp increase in blood plasma levels of the drug. Moreover, widely fluctuating plasma concentrations of the drug may result in the administration of less than therapeutic amounts of the drug in a conservative dosing regimen or amounts too large for the particular patient in an aggressive dosing regimen. Therefore, it is desirable to create a once-daily dosing form

Pregabalin is not uniformly absorbed in the gastrointestinal tract, in the small intestine and ascending colon, but rarely in intestine segments outside the hepatic flexure of the colon. The average absorption window of pregabalin is about 6 hours or less, and thus if pregabalin is prepared in a conventional sustained release formulation, the released drug cannot be effectively absorbed after the formulation passes through the colonic hepatic flexure after more than 6 hours. Therefore, there is a need to develop a sustained release film-coated tablet of pregabalin that can be retained in the stomach for a longer period of time, improve patient compliance and ensure the sustained and stable release of the drug.

### SUMMARY OF THE INVENTION

The present invention provides a sustained release film-coated tablet containing pregabalin or pharmaceutically acceptable salts thereof, that is useful for once-daily (QD) oral dosing. While it is retained in the stomach, the tablet continuously releases pregabalin.

Another embodiment of the present invention provides a sustained release film-coated tablet containing pregabalin or pharmaceutically acceptable salts thereof, and the excipients include at least two release retarding agents, a diluent, and a lubricant.

Further, another embodiment of the present invention involves a process for preparing the sustained release film-coated tablet for oral administration.

Another embodiment of the present invention can effectively treat postherpetic neuralgia, fibromyalgia, and as adjunctive therapy for the treatment of partial-onset seizures in adults. It is also indicated for the management of neuropathic pain related to spinal cord injury and diabetic peripheral neuropathy.

### OBJECTS OF THE INVENTION

The primary object of the present invention is to provide a stable once-daily Sustained release film-coated tablet of pregabalin or pharmaceutically acceptable salts thereof for oral administration.

Another object of the present invention is to provide a therapeutically effective amount of pregabalin or pharmaceutically acceptable salts thereof, hydroxy ethyl cellulose, carbomer (Carbopol 71G), and a combination of polyvinyl acetate, povidone, sodium lauryl sulphate, and silica (kollidon SR) as release retarding agents, and one or more pharmaceutically acceptable excipients.

A further object of the present invention relates to the sustained release film-coated tablet preparation of pregabalin or pharmaceutically acceptable salts thereof, for oral administration once-daily, reducing administration times, and having pharmacokinetic properties of reducing side effects while increasing patient compliance and extending the efficacy time duration.

Yet another object of the present invention provides taste-masking properties such that the administration of the sustained release film-coated tablet is not unpleasant for elderly patients.

### DETAILED DESCRIPTION OF THE INVENTION

The present invention relates to a Sustained release of a film-coated tablet comprising pregabalin or a pharmaceutically acceptable salt thereof, suitable for oral administration.

Sustained release dosage forms are designed to release drugs at a predetermined rate while maintaining a consistent drug level for a specified period of time with the minimum possible side effects. The basic principle behind a sustained release drug delivery system is to optimize the biopharmaceutical, pharmacokinetic, and pharmacodynamic properties of a drug in such a way that its utility is maximized, side effects are reduced and a cure for the disease is achieved. Sustained release drug delivery improves patient compliance by reducing the frequency of drug administration, reducing steady-state fluctuation of drug levels, increasing the safety margin of low therapeutic index drugs, maximizing the utilization of the drug, lowering healthcare costs through improved therapy, and shortening the treatment regimen. Sustained release dosage forms provide sustained drug levels in plasma that frequently eliminate the need for night dosing, which is beneficial for patient compliance.

The term “about”, as and when used in this specification, means ±10% of the mentioned value.

According to one embodiment, the Sustained release film-coated tablet of the present invention comprises pregabalin and release retarding agents. In addition, the Sustained release film-coated tablet further comprises at least one pharmaceutically acceptable excipient selected from a diluent and lubricant. In one embodiment, the pregabalin is present in the range of about 20% w/w to about 80% w/w, preferably in the range of about 30% w/w to about 50% w/w, and is administered as an oral solid dosage form.

According to one embodiment of the present invention, the composition comprises a diluent selected from dextrates, microcrystalline cellulose, dextrose, fructose, Sorbitol, pregelatinized starch, xylitol, sucrose, maltodextrin, maltose, mannitol or combinations thereof. In the present invention, mannitol is preferred as a diluent in the range of about 0.05% w/w to about 60% w/w, preferably in the range from about 0.20% w/w to about 40% w/w. In the development of tablets, the use of mannitol as a filler has increased due to its physicochemical properties, such as its lower hygroscopicity, chemical inertness, and advantageous tableting behavior, including compactability and the ability to produce extremely robust tablets.

According to one another embodiment of the present invention, the composition comprises a release retarding agent selected from the group consisting of hydroxypropyl methylcellulose, hydroxyethyl methylcellulose, hydroxypropyl cellulose, hydroxymethyl cellulose, Hypromellose, Hypromellose acetate succinate, hydroxyethyl cellulose, carboxymethyl cellulose, cellulose acetate, cellulose acetate phthalate, calcium carboxymethyl cellulose, sodium carboxymethyl cellulose, methylcellulose, ethylcellulose, chitosan, gelatin, poloxamer, polyvinyl alcohol, polyvinyl acetate phthalate, polyethylene glycol, polyvinyl pyrrolidone-polyvinyl acrylate copolymer, polyvinyl alcohol-polyethylene glycol copolymer, polyvinyl pyrrolidone-polyvinyl acetate copolymer, kollidon SR, hydroxypropyl starch, magnesium aluminium silicate, polydextrose, polyethylene glycol alginate, carbomer or combinations thereof. In the present invention combination of hydroxyethyl cellulose, carbomer (Carbopol 71G), and the combination of polyvinyl acetate, povidone, sodium lauryl sulphate, and silica (kollidon SR) is preferred as a release retarding agent present in the range of about 2% w/w to about 80% w/w, preferably in the range from about 4% w/w to about 75% w/w. Hydroxyethyl cellulose is present in the range from about 4% w/w to about 40% w/w, preferably from about 8% w/w to about 20% w/w, carbomer (Carbopol 71G) is present in the range from about 1% w/w to about 10% w/w, preferably from about 4% w/w to about 8% w/w. A combination of polyvinyl acetate, povidone, sodium lauryl sulphate, and silica (kollidon SR) is present in the range from about 5% w/w to about 65% w/w, preferably from about 9% w/w to about 50% w/w. A release retarding agent refers to those substances that retard or delay the release of a drug and provide a longer duration of therapeutic response after administration of the dosage form.

According to one more embodiment of the present invention, at least one further pharmaceutically acceptable excipient is a lubricant selected from boric acid, magnesium stearate, Sodium Stearyl fumarate, micronized polyoxy ethylene glycol, leucine, sodium benzoate, sodium acetate, sodium lauryl sulphate, steric acid, sodium stearate, sodium oleate, calcium stearate, waxes or combinations thereof. Magnesium stearate is preferred as a lubricant for the present invention and is present in the range from about 0.15% w/w to about 15% w/w, preferably in the range from about 0.5% w/w to about 5% w/w.

According to one another embodiments of the present invention, the pregabalin sustained release film-coated tablet may further comprise a film coating material such as vinyl polymers like polyvinylpyrrolidone, polyvinyl alcohol and acetate, cellulosics such as methyl and ethyl cellulose, hydroxyethyl cellulose, hydroxypropyl methylcellulose, hydroxypropyl cellulose, acrylates and methacrylates, copolymers such as the vinyl-maleic acid and styrene-maleic acid, and natural gums and resins such as zein, gelatin, shellac, acacia or combinations thereof. In the present invention combination of polyvinyl alcohol, calcium carbonate, polyethylene glycol, and talc (Opadry TF) is preferred as a film coating material.

According to the preferred embodiment of the present invention is to provide a stable once-daily Sustained release film-coated tablet of pregabalin or pharmaceutically acceptable salts thereof, and the excipients include hydroxy ethyl cellulose, carbomer (Carbopol 71G), and a combination of polyvinyl acetate, povidone, sodium lauryl sulphate, and silica (kollidon SR) as release retarding agents. In addition, the Sustained release film-coated tablet further comprises at least one pharmaceutically acceptable excipient selected from a diluent and lubricant.

According to the preferred embodiment of the present invention has been made to solve the above-mentioned problems, and its object is to provide pregabalin containing sustained release film-coated tablet preparation for oral administration once-daily, reduce administration times, and have pharmacokinetic properties of reducing side effects while increasing patient compliance and extending the efficacy time duration, as compared to conventional pregabalin containing preparations for oral administration twice daily. Pregabalin is used for the treatment or prevention of neuropathic pain, epilepsy, fibromyalgia, diabetic peripheral neuropathy, postherpetic neuralgia, or generalized anxiety disorder.

According to one preferred embodiment of the present invention comprising pregabalin or pharmaceutically acceptable salts thereof, present in an amount from about 20% w/w to about 80% w/w, preferably in the range of about 30% w/w to about 50% w/w, the release retarding agent such as hydroxyethylcellulose is present in the range from about 4% w/w to about 40% w/w, preferably from about 8% w/w to about 20% w/w, carbomer (Carbopol 71G) is present in the range from about 1% w/w to about 10% w/w, preferably from about 4% w/w to about 8% w/w. A combination of polyvinyl acetate, povidone, sodium lauryl sulphate, and silica (kollidon SR) is present in the range from about 5% w/w to about 65% w/w, preferably from about 9% w/w to about 50% w/w. Mannitol is preferred as a diluent in the range of about 0.05% w/w to about 60% w/w, preferably in the range from about 0.20% w/w to about 40% w/w. Magnesium stearate is preferred as a lubricant for the present invention and is present in the range from about 0.15% w/w to about 15% w/w, preferably in the range from about 0.5% w/w to about 5% w/w.

According to one preferred embodiment pregabalin is present in an amount of about 165 mg, about 330 mg, or about 660 mg per tablet.

According to another embodiment, 80% of the pregabalin is released in 24 hours, preferably more than 90% is released within 24 hours.

According to one another embodiment of the present invention, Pregabalin, Mannitol, Hydroxy Ethyl Cellulose, Carbopol 71G, and Kollidon SR are sieved separately through a 40 #sieve and magnesium stearate separately through a 60 #sieve. Pre-blending of all ingredients except magnesium stearate for 15 minutes at 20 RPM. Magnesium stearate was added to the mixture and blended for an additional 15 minutes at 20 RPM. The lubricated blend was compressed by a compression machine to form a tablet. The Compressed uncoated tablets are coated in which the coating material is dispersed with purified water in an S.S container under stirring for 45 minutes until the white color suspension is obtained. The tablet is coated at a maximum of 30 °C temperature.

The invention is further illustrated by the following examples, which are by no means intended to limit the scope of the invention but are given by way of illustration.

### Example 1

The Sustained release film-coated tablet was made according to the method defined below using the formulation having the ingredients shown in table I:

TABLE I

| Ingredients | % w/w |
|---|---|
| Pregabalin | 37.29 |
| Mannitol | 30.65 |
| Hydroxy ethyl cellulose | 10.47 |
| (Natrosol 250) | |
| Kollidon SR (Polyvinyl acetate, | 11.63 |
| povidone, sodium lauryl sulfate, | |
| and silica) | |
| Carbopol 71G | 6.63 |
| Magnesium stearate | 1.09 |
| Theoretical Average weight | 100 |
| of the uncoated tablet | |

### Manufacturing Process:

All ingredients were collected and weighed accurately. Pregabalin, Mannitol, Hydroxy Ethyl Cellulose, Carbopol 71G, and Kollidon SR are sieved separately through a 40 #sieve, and magnesium stearate separately through a 60 #sieve. Pre-blending of all ingredients except magnesium stearate for 15 minutes at 20 RPM. Magnesium stearate was added to the mixture and blended for an additional 15 minutes at 20 RPM. Finally, the lubricated blend is compressed to form tablets.

The prepared blended mixture shows the characteristics mentioned in the table below:

| Test Parameters of blend | Result |
|---|---|
| Bulk Density (gm/ml) | 0.424 |
| Tap Density (gm/ml) | 0.583 |
| Carr's Index (%) | 27.27 |
| Hausner's Ratio | 1.38 |

### Example 2

The Sustained release film-coated tablet was prepared according to the procedure described below using the formulation having the ingredients shown in table II:

TABLE II

| Ingredients | % w/w |
|---|---|
| Pregabalin | 37.29 |
| Mannitol | 19.03 |
| Hydroxy ethyl cellulose | 10.47 |
| (Natrosol 250) | |
| Kollidon SR (Polyvinyl acetate, | 23.26 |
| povidone, sodium lauryl sulphate, | |
| and silica) | |
| Carbopol 71G | 6.63 |
| Magnesium stearate | 1.09 |
| Theoretical Average weight | 100.0 |
| of the uncoated tablet | |

### Manufacturing Process:

All ingredients were collected and weighed accurately. Pregabalin, Mannitol, Hydroxy Ethyl Cellulose, Carbopol 71G, and Kollidon SR are sieved separately through a 40 #sieve, and magnesium stearate separately, through a 60 #sieve. Pre-blending of all ingredients except magnesium stearate for 15 minutes at 20 RPM. Magnesium stearate was added to the mixture and blended for an additional 15 minutes at 20 RPM. The lubricated blend was compressed by a compression machine to form a tablet.

| Test Parameters of blend | Result |
|---|---|
| Bulk Density (gm/ml) | 0.438 |
| Tap Density (gm/ml) | 0.588 |
| Carr's Index (%) | 25.63 |
| Hausner's Ratio | 1.34 |

The tablets present the characteristics mentioned in the table below:

| Test Parameters | Test Parameters | Result |
|---|---|---|
| Weight (mg) | 860.00 mg ± 5.0% | 857.14 |
| | (817.00 mg to 903.00 mg) | |
| Hardness (N) | 150N-250N | 124N |
| Thickness (mm) | 5.60 ± 0.30 mm | 5.47 |
| | (5.30 mm to 5.90 mm) | |
| Friability (%) | NMT 1.0% | 0.6 |

### Example 3

The Sustained release film-coated tablet was prepared according to the procedure described below using the formulation having the ingredients shown in table III:

TABLE III

| Ingredients | % w/w |
|---|---|
| Pregabalin | 37.29 |
| Mannitol | 4.93 |
| Hydroxy ethyl cellulose | 10.47 |
| (Natrosol 250) | |
| Kollidon SR (Polyvinyl acetate, | 37.29 |
| povidone, sodium lauryl | |
| sulphate, and silica) | |
| Carbopol 71G | 6.44 |
| Magnesium stearate | 1.06 |
| Theoretical Average weight | 100.00 |
| of the uncoated tablet | |
| Opadry TF (Polyvinyl Alcohol, | 2.82 |
| Calcium carbonate, | |
| Polyethylene glycol, Talc) | |
| Purified Water | Qs. |

The theoretical Average weight of the coated tablet is 885.000 mg

### Manufacturing Process:

All ingredients were collected and weighed accurately. Pregabalin, Mannitol, Hydroxy Ethyl Cellulose, Carbopol 71G, and Kollidon SR are sieved separately through a 40 #sieve, and magnesium stearate separately, through a 60 #sieve. Pre-blending of all ingredients except magnesium stearate was done for 15 minutes at 20 RPM. Magnesium stearate was added to the mixture and blended for an additional 15 minutes at 20 RPM. The lubricated blend was compressed by a compression machine to form a tablet. The Compressed uncoated tablets are coated in which the coating material is dispersed with purified water in an S.S container under stirring for 45 minutes until the white color suspension is obtained. The tablet is coated at a maximum of 30 °C temperature.

| Test Parameters of blend | Result |
|---|---|
| Bulk Density (gm/ml) | 0.449 |
| Tap Density (gm/ml) | 0.592 |
| Carr's Index (%) | 24.15 |
| Hausner's Ratio | 1.31 |

The tablets present the characteristics mentioned in the table below:

| Test Parameters | Test Parameters | Result |
|---|---|---|
| Weight (mg) | 860.00 mg ± 5.0% | 858.50 |
| | (817.00 mg to 903.00 mg) | |
| Hardness (N) | 150N-250N | 160 |
| Thickness (mm) | 5.60 ± 0.30 mm | 5.61 |
| | (5.30 mm to 5.90 mm) | |
| Friability (%) | NMT 1.0% | 0.4 |

### Example 4

The Sustained release film-coated tablet was prepared according to the procedure described below using the formulation having the ingredients shown in table IV:

TABLE IV

| Ingredients | % w/w |
|---|---|
| Pregabalin | 37.29 |
| Mannitol | 0.41 |
| Hydroxy ethyl cellulose | 14.69 |
| (Natrosol 250) | |
| Kollidon SR (Polyvinyl acetate, | 37.29 |
| povidone, sodium lauryl sulphate, | |
| and silica) | |
| Carbopol 71G | 6.44 |
| Magnesium stearate | 1.06 |
| Theoretical Average weight | 100.00 |
| of the uncoated tablet | |
| Opadry TF (Polyvinyl Alcohol, | 2.82 |
| Calcium carbonate, Polyethylene | |
| glycol, Talc) | |
| Purified Water | QS |

The theoretical Average weight of the coated tablet is 885.000 mg

### Manufacturing Process:

All ingredients were collected and weighed accurately. Pregabalin, Mannitol, Hydroxy Ethyl Cellulose, Carbopol 71G, and Kollidon SR are sieved separately through a 40 #sieve, and magnesium stearate separately, through a 60 #sieve. Pre-blending of all ingredients except magnesium stearate was done for 15 minutes at 20 RPM. Magnesium stearate was added to the mixture and blended for an additional 15 minutes at 20 RPM. The lubricated blend was compressed by a compression machine to form a tablet. The Compressed uncoated tablets are coated in which the coating material is dispersed with purified water in an S.S container under stirring for 45 minutes until the white color suspension is obtained. The tablet is coated at a maximum of 30 °C temperature.

| Test Parameters of blend | Result |
|---|---|
| Bulk Density (gm/ml) | 0.467 |
| Tap Density (gm/ml) | 0.609 |
| Carr's Index (%) | 23.33 |
| Hausner's Ratio | 1.30 |

### Example 5

The tablet prepared according to example 4 was subjected to a stability study of 40 °C±2 °C/75% RH±5% RH for 1 month. Results are tabulated below.

Pregabalin 330 mg Sustained release
film-coated tablets (Uncoated Tablet)

| | | Exp 4 |
|---|---|---|
| Test | Specification | (Initial) |
| Average | 860.00 mg ± 5.0% | 861.45 |
| weight | (817.00 mg to 903.00 mg) | |
| Hardness | 150N-250N | 184N |
| Thickness | 5.60 ± 0.30 mm | 5.61 |
| | (5.30 mm to 5.90 mm) | |
| Friability | NMT 1.0% | 0.2 |

Pregabalin 330 mg Sustained release
film-coated tablets (Coated Tablet)

| | | | Exp 4 |
|---|---|---|---|
| | | | (After 1 |
| | | Exp 4 | month at |
| Test | Specification | (Initial) | 40 °C ± 2 °C) |
| Average | 885.00 mg ± 5.0% | 887.24 | 886.78 |
| weight | (840.75 mg to 929.25 mg) | | |
| Thickness | 5.75 ± 0.3 mm | 5.72 | 5.74 |
| | (5.45 mm-6.05 mm) | | |
| Assay | 95.0%-105.0% of the labeled | 98.8 | 99.2 |
| | amount of Pregabalin. | | |

| | Time | % Drug | % Drug | % Drug |
|---|---|---|---|---|
| | (hours) | release | release | release |
| Dissolution | 01 | 10-30% | 21.5 | 16.5 |
| | 04 | 30-50% | 44.6 | 42.2 |
| | 08 | 50-70% | 61.5 | 60.9 |
| | 12 | 60-80% | 72.8 | 73.1 |
| | 24 | Not less than 85% | 93.0 | 95.5 |

1. A Sustained release film-coated tablet adapted for once-daily dosing, comprising pregabalin or pharmaceutically acceptable salts thereof, present in an amount from about 20% w/w to about 80% w/w, preferably in the range of about 30% w/w to about 50% w/w, at least two release retarding agents, film coating material, and one or more pharmaceutically acceptable excipients.

2. The Sustained release film-coated tablet according to claim 1, wherein release retarding agents are present in an amount from about 2% w/w to about 80% w/w, preferably in the range from about 4% w/w to about 75% w/w.

3. The Sustained release film-coated tablet according to claim 1, wherein the release retarding agent is selected from the group consisting of hydroxypropyl methylcellulose, hydroxyethyl methylcellulose, hydroxypropyl cellulose, hydroxymethyl cellulose, Hypromellose, Hypromellose acetate succinate, hydroxyethyl cellulose, carboxymethyl cellulose, cellulose acetate, cellulose acetate phthalate, calcium carboxymethyl cellulose, sodium carboxymethyl cellulose, methylcellulose, ethylcellulose, chitosan, gelatin, poloxamer, polyvinyl alcohol, polyvinyl acetate phthalate, polyethylene glycol, polyvinyl pyrrolidone-polyvinyl acrylate copolymer, polyvinyl alcohol-polyethylene glycol copolymer, polyvinyl pyrrolidone-polyvinyl acetate copolymer, Kollidon SR, hydroxypropyl starch, magnesium aluminium silicate, polydextrose, polyethylene glycol alginate, carbomer or combinations thereof.

4. The Sustained release film-coated tablet according to claim 1, wherein the release retarding agent is hydroxy ethyl cellulose present in the range from about 4% w/w to about 40% w/w, preferably in the range from about 8% w/w to about 20% w/w.

5. The Sustained release film-coated tablet according to claim 1, wherein the release retarding agent is the combination of Polyvinyl acetate, povidone, sodium lauryl sulfate, and silica (kollidon SR), present in the range from about 5% w/w to about 65% w/w, preferably from about 9% w/w to about 50% w/w.

6. The Sustained release film-coated tablet according to claim 1, wherein the release retarding agent is carbomer (Carbopol 71G) present in the range of about 1% w/w to about 10% w/w, preferably from about 4% w/w to about 8% w/w.

7. The Sustained release film-coated tablet according to claim 1, wherein the pharmaceutical composition further comprises one or more pharmaceutically acceptable excipients, selected from the diluent, and lubricant.

8. The Sustained release film-coated tablet according to claim 7, wherein the diluent is selected from dextrates, microcrystalline cellulose, dextrose, fructose, Sorbitol, pregelatinized starch, xylitol, sucrose, maltodextrin, maltose, mannitol or combinations thereof.

9. The Sustained release film-coated tablet according to claim 8, wherein the diluent is mannitol present in the range of about 0.05% w/w to about 60% w/w, preferably in the range from about 0.20% w/w to about 40% w/w.

10. The Sustained release film-coated tablet according to claim 7, wherein the lubricant is selected from the group consisting of boric acid, magnesium stearate, sodium Stearyl fumarate, micronized polyoxyethylene glycol, leucine, sodium benzoate, sodium acetate, sodium lauryl sulfate, stearic acid, sodium stearate, sodium oleate, calcium stearate, or combinations thereof.

11. The Sustained release film-coated tablet according to claim 10, wherein the lubricant is magnesium stearate present in the range from about 0.15% w/w to about 15% w/w, preferably in the range from about 0.5% w/w to about 5% w/w.

12. The Sustained release film-coated tablet according to claim 1, wherein the film coating material is selected from the group consisting of vinyl polymers like polyvinylpyrrolidone, polyvinyl alcohol, and acetate, cellulosics such as methyl and ethyl cellulose, hydroxyethyl cellulose, hydroxypropyl methylcellulose, hydroxypropyl cellulose, acrylates and methacrylates, copolymers such as the vinyl-maleic acid and styrene-maleic acid, and natural gums and resins such as zein, gelatin, shellac, acacia or combinations thereof.

13. The Sustained release film-coated tablet according to claim 12, wherein the film coating material is the combination of polyvinyl alcohol, calcium carbonate, polyethylene glycol, and talc (Opadry TF).

14. The Sustained release film-coated tablet according to claim 1, wherein the pregabalin is present in an amount of about 165 mg, about 330 mg, or about 660 mg per tablet.

15. The Sustained release film-coated tablet according to claim 1, wherein the process comprising;
(a) Sieving pregabalin, mannitol, hydroxy ethyl cellulose, Carbopol 71G, and Kollidon SR separately through a 40 #sieve, and magnesium stearate separately through a 60 #sieve;
(b) Blending of all the above ingredients of step (a) except magnesium stearate for 15 minutes at 20 RPM;
(c) Adding magnesium stearate to the mixture of step (b) and blending for an additional 15 minutes at 20 RPM;
(d) compressing the lubricated blend to the tablet;
(e) coating of tablet with the coating material; and
(f) The tablet is coated at a maximum of 30 °C temperature.

16. The Sustained release film-coated tablet according to claim 1, wherein 80% of the pregabalin or pharmaceutically acceptable salts thereof, is released in 24 hours, preferably more than 90% is released within 24 hours.

17. The Sustained release film-coated tablet according to claim 1, comprising 330 mg pregabalin or pharmaceutically acceptable salts thereof, exhibits a dissolution profile such that after combining the tablet with 900 ml of a 0.06N HCl dissolution medium at 37 °C±0.5. according to USP paddle II, 50 rpm paddle speed,
about 10% to about 30% of the total amount of pregabalin or pharmaceutically acceptable salts thereof, is released at 1 hour;
about 30% to about 50% of the total amount of pregabalin or pharmaceutically acceptable salts thereof, is released at 4 hours;
about 50% to about 70% of the total amount of pregabalin or pharmaceutically acceptable salts thereof, is released at 8 hours;
about 60% to about 80% of the total amount of pregabalin or pharmaceutically acceptable salts thereof, is released at 12 hours; and
about not less than 85% of the total amount of pregabalin or pharmaceutically acceptable salts thereof, is released at 24 hours.

18. The Sustained release film-coated tablet according to claim 1, is for the treatment or prevention of neuropathic pain, epilepsy, fibromyalgia, diabetic peripheral neuropathy, postherpetic neuralgia, or generalized anxiety disorder.
